(19) United States
(12) Patent Application Publication
(10) Pub. No.: US 2002/0177572 A1
(43) Pub. Date: Nov. 28, 2002

(54) Viral infection of cells

(76) Inventors: Marc Feldmann, London (GB); Brian Maurice John Foxwell, Hounslow (GB); Fionula Mary Brennan, Teddington (GB); Jan Bondeson, London (GB)

Correspondence Address: BARNES & THORNBURG, 11 South Meridian Street, Indianapolis, IN, 46204, US

(21) Appl. No.: 10/033,267

(22) Filed: Oct. 25, 2001

Related U.S. Application Data
(63) Continuation of application No. 09/508,350, filed on Apr. 7, 2000, now abandoned.
Application No. 09/508,350 is a 371 of international application No. PCT/GB98/02753, filed on Sep. 11, 1998.

(30) Foreign Application Priority Data
Sep. 11, 1997 (GB) 9719238.9

(51) Int. Cl. A61K 48/00; C12N 15/861

(52) U.S. Cl. 514/44; 435/456; 424/93.21

(57) ABSTRACT

The present invention relates to an improved in vitro method for infecting cells with a viral vector capable of transporting recombinant nucleic acids into the cells. The method comprises the steps of subjecting the cells to elutriation in a velocity gradient, collecting the cells, contacting the cells with at least one cytokine, and infecting 90% or more of the cells with the viral vector at a multiplicity of infection of 50 to 100.

This current invention relates to an improved in vitro method for infecting cells with a viral vector capable of transporting recombinant nucleic acid to those cells. The invention produces an improved level of viral infectivity. The invention also relates to cells infected by such a method.

The use of viruses such as human adenovirus or retroviruses such as Human Immunodeficiency Virus (HIV) derivatives as vectors to deliver exogenous or recombinant nucleic acid, such as foreign genes, into cells is well known. Replication-defective forms of human adenovirus have been used to deliver foreign genes into diverse cells types including liver, muscle, nerve and airway epithelial cells. The success of adenoviruses as a gene deliver; vector is based in part on their highly efficient mode of cell entry and their lack of requirement for host cell replication.

A major difficulty in using viral vectors to transport recombinant nucleic acid into cells has been the difficulty in achieving high levels of infectivity in some cells. Where the level of infectivity and hence gene transfer is low, any effect of the transferred gene an the cells may be difficult to observe as a result of the background of non-infected cells. A high level of gene transfer would allow the study of, for example, intracellular mechanisms, without any requirement to remove a non-infected population of cells. This would allow the regulation of, for example, endogenous chromosomal genes to be studied and precludes the need to use ectopically expressed reporter gene constructs which may be regulated differently. Alternatively, high levels of infection would obviate the need for cloning of infected cells.

When adenovirus is used to infect monocytes, for example, the maximum level of infection achieved has been approximately 50% which required a multiplicity of infection (M.O.I.), as plaque forming units per cell of up to 1000 (Huang, 1995 J. Virol. 69-2257 Haddada., 1993, Biochem. Biophys. Res. Commun. 195:1174-83). Typically monocytes are prepared by isolating mononuclear cells from healthy adult donors using density gradients, washing the mononuclear cell fraction by low-speed centrifugation and resuspending the mononuclear cells obtained in growth medium. The cells are then allowed to adhere to tissue-culture flasks. The adherent cell population comprises typically 90% monocytes. The monocytes obtained may then be treated with a cytokine, such as a granulocyte-macrophage colony stimulating factor (GM-CSF) or macrophage colony stimulating factor (M-CSF), prior to infection with the viral vector. These cytokines increase the expression of integrins (αVβ3 and αVβ5) on the cell surface and are required for viral entry into cells.

The inventors have identified an improved method of infecting cells with viral vectors which produces infectivity rates of approximately 100% with considerably lower concentrations of viral vector. This removes the necessity to remove non-infected cells and means that less recombinant viral-vector need be used. Consequently the regulation of cellular pathways can be studied.

The inventors realised that inflammatory diseases such as rheumatoid arthritis, respiratory diseases and inflammatory diseases of the bowel are manifest as a result of the activity of activated monocytes which are likely to express high levels of integrins, aVβ3 and aVβ5. Such cells have not previously been infected with viral vectors. The inventors have demonstrated that such cells may be successfully infected with adenovirus. This allows biochemical mechanisms within cells to be selectively studied and new targets for therapeutic intervention to treat the disease to be identified.

This also means that such diseases may be treated by gene therapy. Adenovirus is known to be used in gene therapy but this is the first time that the use of adenovirus to treat inflammatory disease has been identified.

Accordingly, the invention provides an in vitro method for infecting one or more cells with viral vector capable of transporting exogenous or recombinant nucleic acid into the cells to be infected, comprising the steps of:

a) collecting the cells to be infected;

b) treating the collected cells with at least one cytokine; and

c) infecting the collected cells with a viral vector.

Preferably the cells are subjected to elutriation prior to collection.

Preferably centrifugal elutriation is used. This combines centrifuging a sample to sediment out particles with elutriation, the process of separation by washing. Typically a suspension of cells is pumped into a funnel shaped chamber at a preset flow rate. As fluid travels through the chamber its velocity decreases as the chamber gets wider thus creating a velocity gradient from the narrow end of the chamber to its widest part. Cells migrate to positions in the velocity gradient where the effects of both the centrifugal force field and the fluid velocity are balanced. Smaller cells are at equilibrium, at the elutriation boundary where the centrifugal force field and the velocity are low. Larger cells will remain near the inlet to the chamber where the centrifugal force field and the velocity are high. By gradually increasing the flow rate, cells can be washed out according to size.

Cytokines have been found to increase the concentration of integrins, such as αVβ3 or αVβ5, on the cell surface. The inventors realised that rheumatoid synovial cells have such integrins in high levels on their cell surface. They have therefore found that viral vectors may, unexpectedly, be infected into rheumatoid synovial cells in high concentrations. This allows the mode of action of pathways within such cells to be modified by the insertion of exogenous nucleic acid into the cells.

Accordingly, a further aspect of the invention provides an in vitro method for infecting one or more rheumatoid synovial cells with a viral vector capable of transporting exogenous or recombinant nucleic acid into the cells to be infected comprising the steps of

a) collecting the rheumatoid synovial cells; and

b) infecting the collected cells with a viral vector.

Other cells obtainable from, for example, brocheoalveolar lavage from inflammatory diseases, such as asthma and chronic obstructive pulmonary disease (C.O.P.D.), and gut biopsies of inflammatory bowel diseases such as Crohn's disease and ulcerative colitis, may also be infected by the method of the invention.

Preferably the viral vector is an adenovirus, especially a replication-deficient adenovirus.

The M.O.I., expressed as plaque forming units per cell, may be 10-1000, preferably 35-100, especially 50-100.

Preferably the cells to be infected are monocytes. Such cells may be isolated from, for example, single donor plateletphoresis blood residues. The cells may be partially purified by density centrifugation to provide the suspension of cells prior to elutriation.

The isolated cells may be cultured in a suitable growth medium, but are preferably treated with cytokine substantially immediately after collection from the elutriator with cytokine.

Cytokine is preferably added to the medium in which the cells are cultured and may be incubated with the cells for between 24 and 96 hours, typically 65-75 hours, preferably about 72 hours. The cytokine may be M-CSF. The concentration of cytokine used is preferably 100 ng-1 μg/ml.

Preferably the exogenous or recombinant nucleic acid within the viral vector is DNA. The recombinant nucleic acid preferably comprises a sequence encoding a gene product of interest operably linked to suitable regulatory sequences to enable the gene to be expressed within the infected cell.

Preferably the foreign gene encodes a modulator for one or more biochemical pathways within the cell to allow the cell pathways to be studied. An example of a modulator is the IκB protein, an endogenous regulator, especially IκBα, an inhibitor of NFκB. Alternatively a mutated enzyme, such as one in which catalytic activity has been destroyed, (a dominant negative construct) may be used.

Alternatively, a single chain antibody or antibody fragment, or antisense DNA may be encoded by the foreign gene.

The invention also relates to cells infected by virus by means of the methods of the invention.

The invention will now be described by way of example only with reference to the following figures:

### BRIEF DESCRIPTION OF THE DRAWINGS

FIG. 1—Differential induction of integrins αVβ5 on monocytes by GM-CSF and M-CSF. Monocytes were untreated or treated with GM-CSF (500U/ml), or M-CSF (1 μg/ml) or both cytokines for 48 hours. The cells were then analysed by FACS for the expression of aVβ3 (broken line) or αVβ5 (dotted line), staining with control Ig (solid line) is also shown.

FIG. 2—The effect of cytokine treatment on adenoviral infection of primary human monocytes. Monocytes were treated with GM-CSF (500U/ml) or M-CSF (1 μg/ml) for 72 hours followed by infection with β-galactosidase (shown by dotted line) containing adenovirus or control virus (solid line) at 100 m.o.i. (a) for various m.o.i. (b). After 72 hours cells were analysed by FACS for expression of β-galactosidase.

FIG. 3—M-CSF treatment is not required for infection of RAW 264.7 cells. RAW 264.7 cells were incubated with or without M-CSF (1 μg/ml) for 48 hours followed by infection with control adenovirus or a vector containing β-galactosidase. Following culture for 4 days the cells were assayed for β-galactosidase expression by FACS. Key: control virus, no M-CSF (thick line), control virus +M-CSF (thin line), β-galactosidase virus, no M-CSF (dashed line), β-galactosidase virus +M-CSF (broken line).

FIG. 4—Expression of IκBα and IκBα adenovirus infected monocytes and RAW 264.7 cells. RAW 264.7 cells (a) or M-CSF treated monocytes (b) were untreated (un) or infected with IκBα adenoviral vector at the given m.o.i. After 72 hours cell lysates and post-nuclear supernatants were analysed for IκBα expression by immunoblotting Equivalent amounts of protein were loaded on each track for the given cell type (c). Nuclear lysates were prepared from M-CSF treated human monocytes that had been infected with IκBα adenovirus or control virus at the given m.o.i. for 72 hours. IκB expression was determined as above: equivalent amounts of protein were loaded an each track.

FIG. 5—Expression of NFκB and IκB proteins in virus infected monocytes. Monocytes were treated with M-CSF for 48 hours. The cells were then infected with a control virus or an IκBα containing adenoviral vector at a m.o.i. of 50. A third group were untreated. Following a further 3 days in M-CSF the cells were washed and half of each group was treated with 10 ng/ml LPS for 30 minutes. The cells were then lysed and cytosol and nuclear extracts were prepared which were then analysed for IκBα/β expression (cytosol) or p65/p50 expression (nuclear) by immunowestern blotting. 150 μg protein and 50 μg protein was analysed for each cytosol or nuclear sample respectively.

FIG. 6—Inhibition of NFκB function in IκBα virus infected monocytes. Monocytes were uninfected or infected with IκBα or control adenovirus followed by LPS (10 ng/ml) stimulation. Nuclear extracts were prepared and 20 μg protein analysed for NFκB (upper panel) or AP-12 (lower panel) activity by EMSA.

FIG. 7—IκBα virus-infection inhibits LPS-induced TNF mRNA and protein. M-CSF treated cells were untreated or infected with IκBα virus or control virus at various m.o.i. After three days cells were harvested and stimulated with LPS (10 ng/ml). (a) After 2 hours cells were harvested and mRNA extracted and analysed for mRNA by Northern blotting with the indicated probes or (b) after 24 hours the culture supernatants were removed and assayed for TNFα by ELISA, the cells were examined for any toxic effect by MTT assay. Data is given as percentage TNF production of non-infected control cells. The results represent the combined data of cells from five different donors (=S.D.). No significant toxicity was observed as judged by the MTT assays (not shown). (c) Cells from rheumatoid synovium were cultured in 48 well plates at 1×10 cells and infected with either Adv1κBα or Adv0 at a m.o.i., of 40 to 1. The supernatants were taken for ELISA assay at 48 hours; pooled data from the 5 patients is shown (+/− s.d.). The null hypothesis that there was no change in TNF production whether cells were infected with Adv1κBα or Adv0 could be rejected (p<0.05) using a Wilcoxon's signed rank test on paired differences compared with untreated cells on each patient.

FIG. 8—Effect of Adv1κBα infection on LPS induced IL-1, IL-6 and IL-8 expression by human macrophages. Effect of infection of human monocyte-derived macrophages with various titers of Adv1κBα or Adv0 on the LPS-induced production of IL-1β (A)2 IL-8 (B) and IL-6 (C), expressed as a percentage of production of the cytokine in question induced by LPS (10 ng/ml) in uninfected cells. Error bars indicate standard error of the mean (n=7-10).

FIG. 9—Effect of exogenous TNF on IL-1b and IL-6 production. Pretreatment with 20 ng/ml or 100 ng/ml TNFα abrogates the inhibitory effect of AdvIκBα infection on LPS-induced IL-1β production (A). TNFα-induced (20 ng/ml) IL-6 is potently inhibited by AdvIκBα infection (B), but TNFα-induced IL-1 is only moderately affected (C). Error bars indicate standard error of the mean (n=6-8).

FIG. 10—Inhibition of NF-kB has only minor effects on IL-10 production. Effect of infection of human monocyte-derived macrophages with various titers of AdvIκBα or Adv0, on IL-10 production induced by LPS (10 ng/ml), expressed as a percentage of IL-10 production induced by the same stimulus in uninfected cells (A). In (B), pretreatment with 20 ng/ml TNFα abrogates the inhibitory effect of AdvIκBα infection on LPS-induced IL-10 production (pg/ml). Error bars indicate standard error of the mean (n=7-10).

FIG. 11—Effect of IκBα infection on IL-1ra production. Effect of infection of human monocyte-derived macrophages with various titers of AdvIκBα or Adv0 on the LPS-induced production of IL-1ra, expressed as a percentage of the production of the same cytokine in uninfected cells challenged with the same stimulus (A). Pretreatment with IL-1, or TNFα (20 ng/ml) alone does not affect, but treatment with both cytokines moderately reverses the inhibitory effect of AdvIκBα infection on the LPS-induced production of IL-1ra (B). Error bars indicate standard error of the mean. n=7-8.

FIG. 12—Adv1κBα infection inhibits LPS-mediated soluble TNF receptors. Effect of infection of human monocyte-derived macrophages with various titers of Adv1κBα or Adv0 on the LPS-induced production of the p55 (A) and p75 (B) soluble TNF receptors, expressed as a percentage of the production of the same cytokine in uninfected cells challenged with the same stimulus. Error bars indicate standard error of the mean, n=7-8.

FIG. 13—Inhibition of NF-KB selectively inhibits TNFα mRNA. Infection with 40:1 of Adv1κBα, but not 40:1 of Adv0, inhibits TNFα mRNA expression (upper panels) induced by PMA (A) or UV light (B) as assessed by Northern, analysis. Zymosan-induced TNFα mRNA expression (C) is unaffected by infection with 80:1 of Adv1κBα. Lower panels contain GADPH expression. This is a representative of three complete experiments.

FIG. 14—PSI inhibits LPS-mediated TNFα where not affecting zymosan-induced TNFα. Effect of the proteosome inhibitor PSI on LPS-induced (▪) and zymosan-induced (●) TNFα production. Error bars indicate standard error of the mean, n=6-7.

FIG. 15—In excess of 90% of rheumatoid synovial cells can be infected with adenovirus. β-galactosidase activity in total rheumatoid synovial cell cocultures (A), or with T lymphocyte (B), macrophage (C) or synoviocyte (D) sub-populations detected through double gating for size/granularity and cell surface markers, in cultures infected with 40:1 of either Adv0 (filled line) or ADVβgal (solid line). A representative of five experiments. In (E), the means of β-galactosidase-positive cells are plotted +/− standard error of the mean, n=5.

FIG. 16—Adv1κBα infection inhibits the spontaneous production of proinflammatory cytokines from rheumatoid synovial cells. Effect of infection of rheumatoid synovial cells with 40:1 of either Adv0 or Adv1κBα on the spontaneous production of IL-1 (A), IL-8 (B) and IL-6 (C). Error bars indicate standard error of the mean, n=6.

FIG. 17—IκBα overexpression permanently inhibits TNFα and IL-6 production. Effect of infection of rheumatoid synovial cells with 40:1 of either Adv0 or Adv1κBα on the spontaneous production of TNFα (A) or IL-6 (B) over time. Symbols denote uninfected ( ), Adv0-infected ( ) or Adv1κBα-infected ( ) cells. A representative of three independent experiments.

FIG. 18—Inhibition of NF-kB through AdvIκBa infection has only marginal effects on the spontaneous production of anti-inflammatory mediators. Effect of infection of rheumatoid synovial cells with 40:1 of either Adv0 or AdvIκBα on the spontaneous production of IL-10 (A), the IL-1 receptor antagonist (B) and the p75 soluble TNF receptor (C). Error bars indicate standard error of the mean, n=6.

FIG. 19—IκBα overexpression potently inhibits MMP-1 and MMP-3 production, but slightly potentiates the production of TIMP-1. Effect of infection of rheumatoid synovial cells with 40:1 of either Adv0 or AdvIκBα on the spontaneous production of MMP-1 (A), MMP-3 (B) and TIMP-1 (C). Error bars indicate standard error of the mean; n=6.

FIG. 20—Effect of Adv1κBα infection on the balance between MMP production versus the production of TIMP-1 over time. Effect of infection of rheumatoid synovial cells with 40:1 of either Adv0 or AdvIκBα on the spontaneous production MMP-1 (A), MMP-3 (B) and TIMP-1 (C) over time. Symbols denote uninfected-, Adv0-infected” or AdvIκBα-infected ▪ cells. A representative of three independent experiments.

### EXAMPLES

### 1. INFECTION OF HUMAN MONOCYTE-DERIVED MACROPHAGES MATERIALS AND METHODS

Isolation of Peripheral Blood Monocytes.

Single donor plateletphoresis blood residues were purchased from North London Blood Transfusion Service (Colindale UK). Mononuclear cells were isolated by Ficoll-Hypoque centrifugation (specific density 1.077 g/ml) preceding monocyte separation in a Beckman JEL elutriator.

Cells were usually collected from the Ficoll gradient resuspended in Hanks and centrifuged @2000 rpm in a centrifuge for 10 minutes. The pellets were resuspended to 50 ml in a Falcon tube and respun @1000 rpm for 15 minutes.

The resulting pellet was resuspended in RPMI with 10% FCS up to 50 ml before injecting into the elutriator. A maximum of 1000×106 cells were loaded into the chamber. Typically the cells were elutriated according to the manufacturer's instructions.

The elutriator head was assembled and 200 ml 1% Etoxaclean was flushed through. This was followed by 1.5L of sterile distilled water. 300 ml of elutriation buffer (RPMI with P/S and 1% low endotoxin FCS) was then pumped through. The sample containing cells to be purified was then aspirated through the elutriator at 2000 rpm and 10OC. Fractions containing the cells of interest were collected in Falcon tubes.

Monocyte purity was assessed by flow cytometry using directly conjugated anti-CD45 and anti-CD14 antibodies (Leucocyte, Becton Dickinson, UK) and was routinely greater than >90%. All media used in separation and culture of monocytes was tested for endotoxin using the Limulus amoebocyte lysate test (Bio Whittaker Inc., Bethesda Md.) and were rejected if endotoxin contamination exceeded 0.1 unit/ml.

### Recombinant Adenovirus Vectors

The recombinant replication-deficient adenovirus vectors encoding E.coli β-galactosidase or having no insert (rAd) was provided by Dr. A. Byrnes (Oxford UK). A second virus encoding E.coli β-galactosidase gene (Ad/β-gal) [Watanabe, 1996, Blood 87:5032-9] was generously provided by Canji, Inc. (San Diego Calif.) and the vector encoding IκBα (rAD IκB) [Wrighton J. Exp. Med. 183:1013-221 by Dr. de Martin (Sandoz, Vienna Austria). The viruses were prepared, purified, titered as previously described [Watanabe, supra]. Virus was produced in the 293 human embryonic kidney cell line and purified by ultracentrifugation through two caesium chloride gradients. The titers of viral stocks were determined by a limiting dilution plaque assay on 293 cells, and were 2.9 to 5.8×1010 infectious units/ml as measured before dilution for use. Viruses were suspended in a buffer solution of 2% sucrose and 2mmol/L MgCl2 and stored at −70° C.

### Cytokine Treatment and Adenoviral Infection of Monocvtes

Human monocytes or murine RAW 264.7 were cultured at 5×105/ml in RPM1 1640 (Bio Whittaker inc.) supplemented with 5% (v/v) heat inactivated foetal calf serum containing 10 units/ml penicillin/streptomycin and 2 mM glutamine at 37° C. Monocytes were treated with GM-CSF (5U/ml) or M-CSF (1 μg/ml) for 72 hours. Expression of integrins was assayed by indirect immunofluorescence and FACS analysis using monoclonal antibodies to αVβ3 (100 μg/ml LM609, provided by IXSYS Corp. (San Diego Calif. USA), or αVβ5 (undiluted active supernatant kindly provided by J. Gamble Smith). The commercially available monoclonal antibody OX14(100 μg/ml) was used as a negative control.

Following cytokine treatment monocytes were washed in RPM 1640 and resuspended at 5×105 cells/ml. The cells were infected with virus at the indicated plaque forming units per cell (multiplicity of infection—m.o.i.). β-galactosidase expression in individual cells was measured by FACS.

### Preparation of Cytosolic Proteins

4×106 cells were plated on petri dishes (Nunc), and cultured overnight. They were stimulated, washed in ice cold PBS, removed from dishes by scraping, and lysed (20 mM Tris pH 8.0, 137 mM NaCl, 1 mM MgCl2, 0.1% NP-40 10 minutes at 4° C.) . Lysates were spun (1200×g 10 minutes 4° C.) and the supernatant was retained and assayed for protein concentration by the commercially available BCA method.

### Preparation of Nuclear Proteins

Nuclear extracts were prepared as previously described [Whiteside, 1992, Nuc. Acids Res. 20:1531-8]. Briefly cells were washed with ice cold PBS then spun (1200×g 30 second 4° C.) and the aspirated pellet was resuspended in low salt lysis buffer (10 mM Hepes, 1.5 mM MgC12 10 m1M KCl 0.5 mM DTT 1 mM PMSF 10μg/ml aprotinin 30 μg/ml leupeptin) at 4° C. for 5 minutes, NP-40 was added to a final concentration of 0.125% and cells were vortexed immediately. The samples were spun (1200× g 10 minutes 4° C.) , the cytosolic fraction was removed and the nuclear pellet was resuspended in nuclear extraction buffer (5 mM Hepes 25% w/v glycerol, 0.4M NaCl 1.5 ml MgC12 0.2 mM EDTA 1 mM DTT 1 mM PMSF 10 μg/ml aprotitin, 10 μg/ml pepstatin 30 μg/ml leupeptin) and left at 4° C. for 1 hour. The samples were spun (1200× g 10 minutes 4° C.) and the supernatant was retained and stores at −70° C.

### Western Blotting of Proteins

Samples were mixed with an equal volume of 2× gel sample buffer (62.5 mM Tris pH 6.8, 2% SDS, 5% β-mercaptoethanol, 10% glycerol, 0.002% bromophenol blue) and boiled for 3 minutes. Equal amounts of protein were loaded in each lane, separated by SDS-PAGE on a 10% w/v Polyacrylamide gel and transferred to nitrocellulose (Boehringer Mannheim). Membranes were blocked with a 5% milk powder solution (20 mM Tris 150 mM NaCl pH 8.0.0.1% Tween 20) and then proteins were detected with rabbit polyclonal antibodies and horseradish peroxidase conjugated donkey anti-rabbit F(ab)2 fragments (Amersham UK), both diluted in milk powder, and visualised by enhanced chemiluminescence (Amersham UK).

### Electrophoretic Mobility Shift Assays

EMSA's were performed as previously described (Clarke, 1995, Eur. J. Immunol. 25: 2961-6). Data was analysed using a Biorad GS-670 densitometer.

Measurement of TNFα production.

RAW 264.7 cells were resuspended at 2×105/ml and plated on 24 well plates at 500 ml per well. They were incubated for 16 hours in the presence or absences LPS (10 ng/ml) and with various concentrations of cytokines. The supernatants were assayed for TNF levels by WEHI 164 (Clone 13) bioassay (Espevik, 1986, J. lmmunol. Methods 95: 99-105).

### Preparation of Cytosolic mRNA

15×106 cells were plated overnight, stimulated for 2 hours with LPS and cytokine, washed in ice cold PBS and resuspended in 400 μl lysis buffer (10 mM tris pH 7.9 150 mM NaCl 1.5 mM MgCl2 0.65% NP-40 10 minutes 4o C. Lysates were spun (12000 ×g, 5 minutes, room temp.). EDTA (1 μl 0.25M) and SDS (20 μl 10% in H₂O) were added to the required supernatant and the tube was gently mixed. mRNA was purified by extraction, with 1:1 phenol chloroform, and precipitation (−20° C., 30 minutes) with sodium acetate (pH 5.2) to a final concentration of 0.3M and 2 columns of ethanol. The precipitate was spun down (1200 ×g, 15 minutes, 4° C.), washed with freezing 70% ethanol, then re-dissolved in water.

### Northern Blotting

mRNA (10 μl, 1 μg/μ1) was mixed with 2 μl H₂O, 5 μl 10× northern buffer (200 mM MOPS, 50 mM sodium acetate), 10 mM EDTA pH 7.0), and 25 μl deionised formamide and heated to 60° C. for 5 minutes. 10 μl 6× loading buffer (0.25% bromophenol blue, 0.25% xylenol cyanol 25% glycerol) was added and the samples were run on an agarose gel (1% agarose, 20 mM MOPS, 5 mM sodium acetate, 1 mM EDTA, 6.5% formaldehyde pH 7.0, 100 mA 4 hours). mRNA was transferred to a Hybond-N membrane (Amersham UK) by capillary action and fixed by baking (80° C., 2 hours). The membrane was pre-incubated in hybridisation solution (50% formamide, 5× SSC, 0.05M sodium phosphate pH 6.6 0.5× Denhardts solution 0.1 mg/ml salmon sperm DNA) for 4 hours at 42° C. and hybridised (12 hours, 42° C.) with 1-3× 106 cpm/ml of 32 p labelled cDNA probe dissolved in hybridisation solution. The membrane was washed in 0.2×SSC 0.1% SDS at 42° C. and exposed to hyperfilm MP at −70° C. for 1-3 days.

### RESULTS

### Efficient Gene Transfer into Primary Macrophage Following Treatment with M-CSF

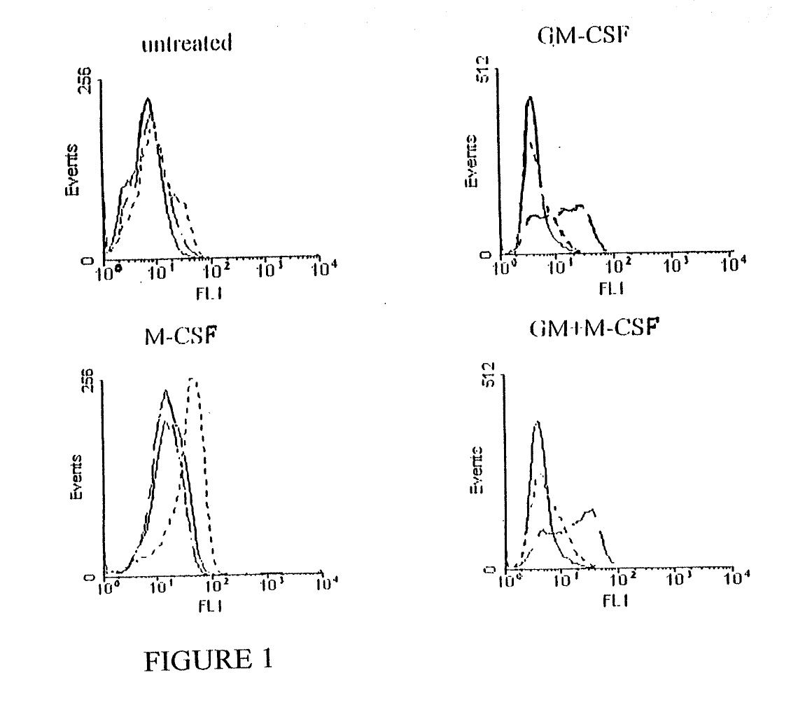

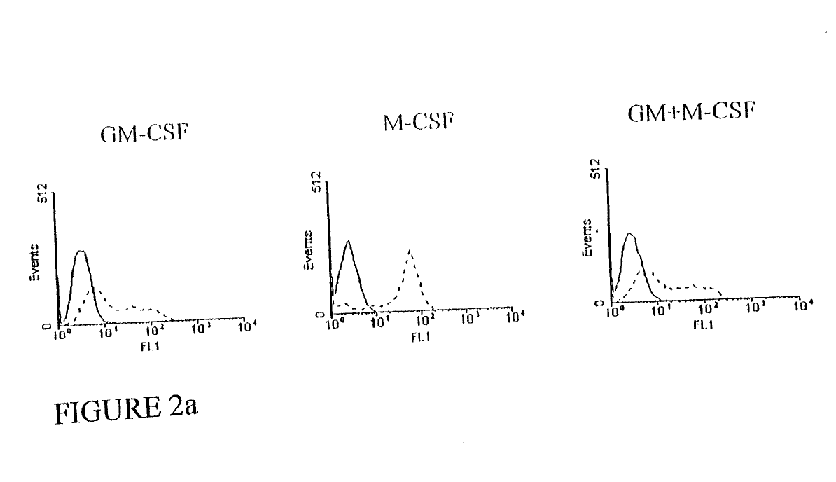

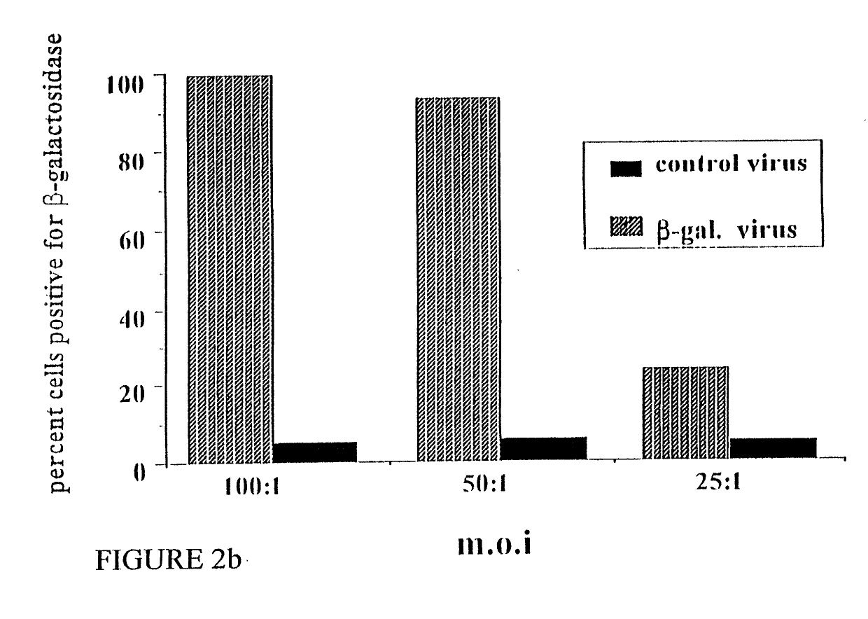

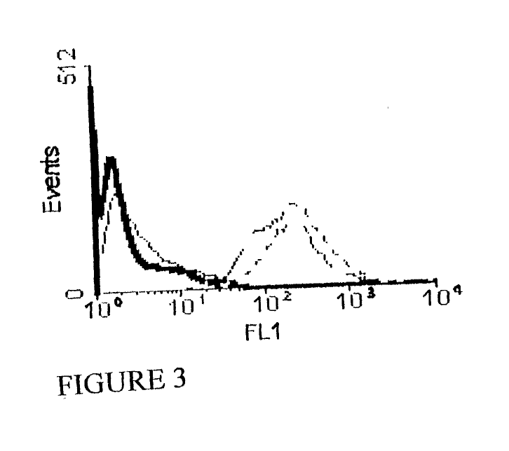

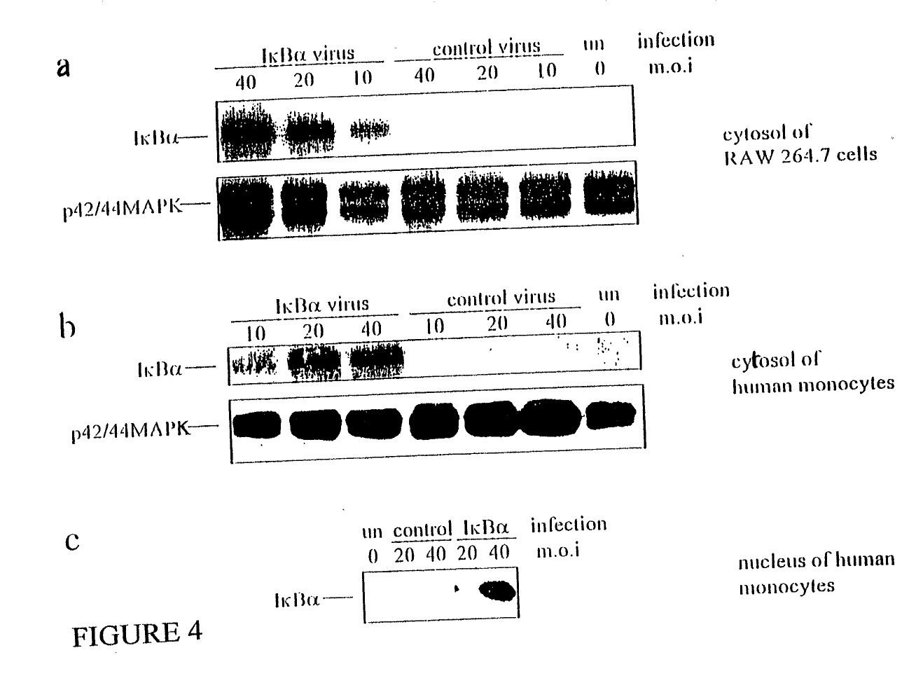

Using an adenoviral vector encoding the E.coli β-galactosidase gene the effectiveness of this approach to transfer genes into primary human monocytes obtained from peripheral blood was investigated. As previously shown by others (Haddad, supra, Huang, Supra) using freshly prepared monocytes the inventors were unable to detect expression of β-galactosidase in these cells following exposure to the virus even at m.o.i.>1000 (results not shown). It has been previously reported that αV integrins are required as cofactors for virus infection [Huang, 1995 supra]. Expression of αVβ3 and αVβ5 was found to be low on resting monocytes (FIG. 1), however treatment with GM-CSF or M-CSF for 48 hours unregulated the expression of αVβ3 or αVβ5 respectively (FIG. 1). Treatment with a combination of both cytokines resulted in integrin expression that resembled GM-CSF alone (FIG. 1). With reference to these observations, the infectivity of monocytes with adenovirus was re-examined. Prior treatment with GM-CSF or M-CSF for 72 hours, which was found to be the optimal treatment, respectively resulted in 52% or 90% of the cells expressing the β-galactosidase gene following treatment with virus at 100 m.o.i. (FIG. 2). As with integrin expression, treatment with the combination of cytokines gave a result (52% of cells positive) similar to GM-CSF alone (FIG. 2). Further studies were therefore pursued using only M-CSF. Reducing the m.o.i. to 50 from 100 had only a small effect on efficiency of infection. 92% compared to 98% (FIG. 3); however at 25 m.o.i. infectivity was greatly reduced (20%). Infection of monocytic/macrophage cell line was also investigated. The human cell lines U937 and THP-1 were totally refractory to infection by adenovirus even following treatment with GM-CSF or M-CSF, neither cytokine was able to induce αVβ3 or αVβ5 expression in these cell lines (results not shown). In contrast, the murine macrophage cell line RAW 264.7 was infected by adenovirus β-galactosidase to a degree similar to human monocytes at 100 m.o.i. (FIG. 4), and still-showed >60% infection at m.o.i. of 10 (results not shown). These cells did not require prior exposure to M-CSF (FIG. 4).

### Adenoviral Transfer of IkBa into Primary Monocvtes

The success of the gene transfer experiments on human monocytes led the inventors to investigate the use of this system for the introduction of genes that would modify important intracellular signalling pathways. For this purpose the inventors used an adenoviral vector expressing IκBα [Baeuerle, 1996, Cell 97:13-20] under the control of CMV promoter, rAD.IκB [Wrighton, Supra].

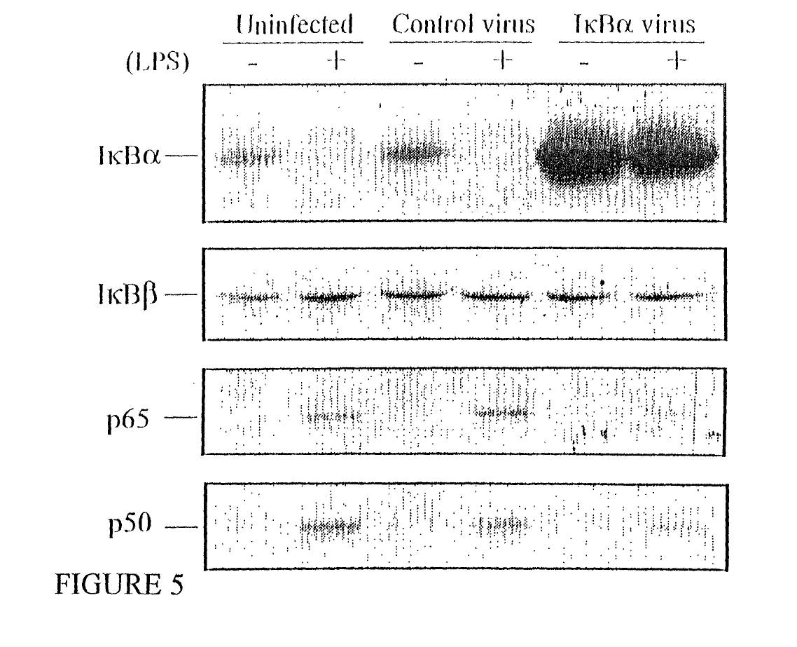

IκB is known to inactivate NFκB a transcription factor within cells. Inhibition of NFκB allows the suppression of proteins whose expression is dependent on the factor to be studied. Infection of both RAW 264.7 cells of M-CSF treated human primary macrophages result in the prominent over-expression of IκBα (FIG. 5). No changes in IκBα expression were evident following infection with control virus. The anti-IκBα antibody used did not recognise murine IκB α, hence the failure to detect endogenous IκBα in the RAW 264.7 cells. As a control, Western blots were reported for p42MAPK that showed no changes to expression following adenovirus infection (FIG. 5). As the IκBα construct also contained a nuclear localisation sequence, the presence of nuclear IκBα could also be detected (FIG. 5c).

### Infection with rAd.IkB Inhibits NFKB Activity in Human Macrophazes

Infection with the virus resulted in the expression of high levels of IκBα even after LPS treatment, unlike uninfected cells or cells infected with control virus (FIG. 5), where IκBα was degraded. Studies on nuclear extracts from the same cells showed that nuclear translocation of NFκB p65 subunit, and to a lesser extent the p50 subunit, was inhibited in virus infected cells. There was also some inhibitory effect on p50 nuclear translocation in cells infected with control virus; the reasons for this are unclear. Unlike IκBα, the levels of IκBα expression were not effected by viral infection. No LPS-induced degradation of IκBα (FIG. 5b) was observed, a finding the inventors have also consequently observed in human monocytes (results not shown).

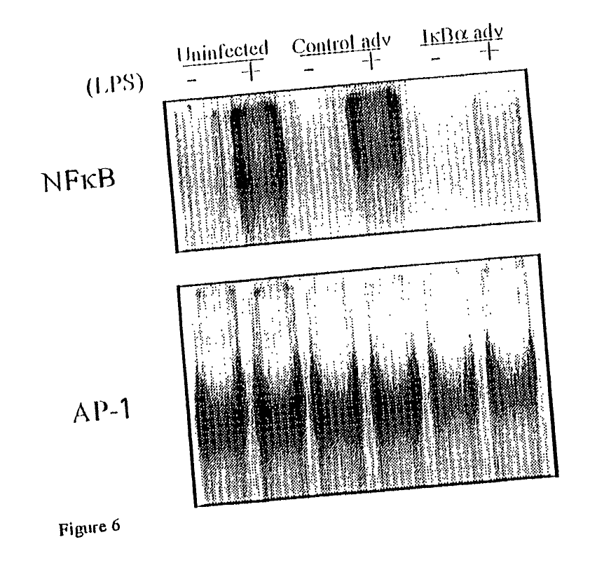

The effect of IκBα virus infection on nuclear NFκB DNA bind activity was also investigated by EMSA (FIG. 6). Infection of monocytes with the virus resulted in total ablation of nuclear NFκB DNA binding activity. No such effect was observed in uninfected or control virus treated cells. The activity of AP-1 was also investigated. Unlike NFκB, AP-1 appeared to be constitutively active in the M-CSF treated cells and no LPS induced activity was detectable. However it was noted that there was a some low level reduction in nuclear AP-1 binding activity in IκBα virus infected cells compared with the control cell populations.

### Infection of Monocytes with IkBa Adenovirus Inhibits LPS Induced TNF Expression

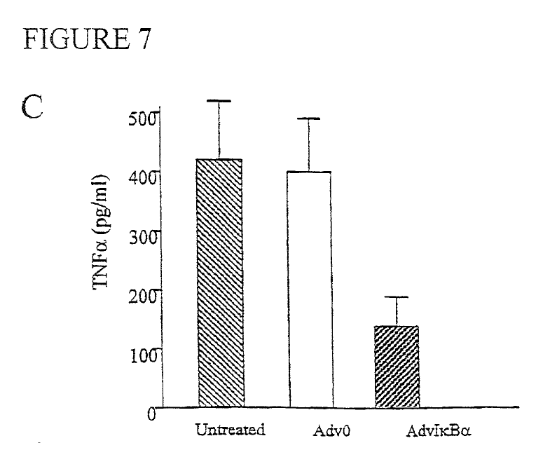

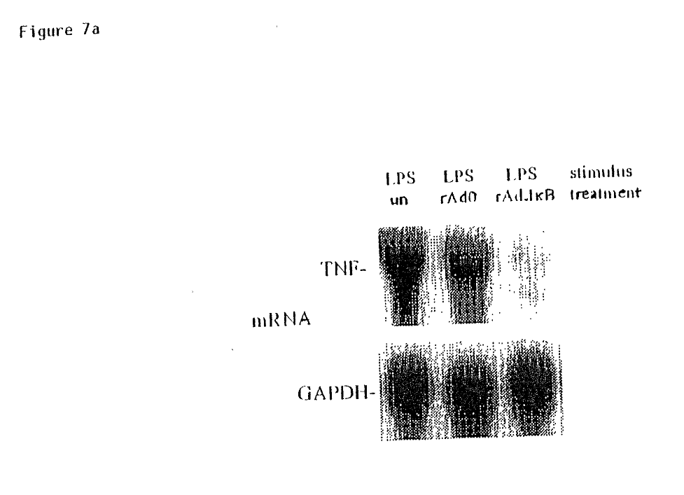

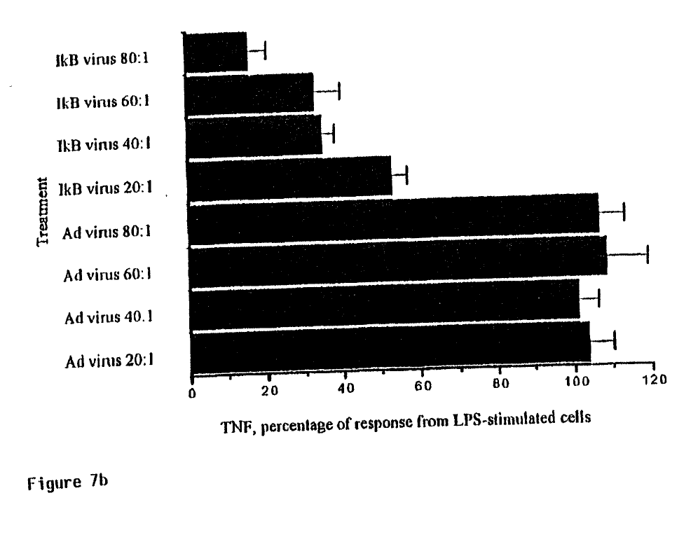

Activation of NFκB is thought to be essential for the expression of TNFα although this hypothesis has never been tested in primary human cells. Infection of monocytes with IκBα virus resulted in inhibition of LPS induced TNFα mRNA expression (FIG. 7a). No effect was observed on the levels of GADPHα mRNA. A similar effect was also observed on the expression of TNFα protein where a clear close dependency relating to the m.o.i. was observed (FIG. 7b). The IκBα virus was also used on synoviocytes obtained from rheumatoid joints. These cells constitutively express TNF and other cytokines. Infection with virus resulted in the inhibition of TNF production by these rheumatoid synovial cells (FIG. 7C).

### Lack of Apoptosis using IkB

As NFκB has been reported to be a key anti-apoptotic mechanism and that inhibition of NFκB induced by TNF resulted in cell death, it was possible that cell depletion may account for the inhibitory effects of the IκB virus. However the inventors failed to observe a toxic effect of the virus at the m.o.i. used, and furthermore treatment of IκBα adenovirus infected macrophages did not undergo apoptosis following treatment with exogenous. TNF (results not shown).

### DISCUSSION

The results show that the method of the invention produces a high efficiency of infection in cells, such as human primary macrophages by adenovirus.

The inventors have demonstrated approximately 100% infection with m.o.i. of 50-100. Previous studies [Haddada 1993 supra and Huang, 1995 supra] only obtained approximately 50% infection which required a m.o.i. of 1000. The inventor's observation that high levels of gene transfer can be achieved with the conditions of the invention has important consequences for the study of intracellular mechanisms of macrophages as it precludes any requirement to remove a non-infected population and obviates the need for cloning. This allows the regulation of endogenous chromosomal genes to be studied and precludes the need to use ectopically expressed reporter constructs which may be regulated differently.

High levels of infectivity of adenovirus allowed the insertion of genes expressing IκBα intracellularly to study its effect on the NFκB to TNFα synthesis to be successfully studied.

### 2. DEMONSTRATION OF SELECTIVE REGULATIONS OF CYTOKINE INDUCTION MATERIALS AND METHODS

### Isolation of Peripheral Blood Monocytes

Single donor plateletphoresis residues were purchased from North London Blood Transfusion Service (Colindale UK). Mononuclear cells were isolated by Ficoll-Hypaque centrifugation preceding monocyte separation in a Beckman JEL elutriator. Monocyte purity was assessed by flow cytometry and was routinely greater than 90%.

### Adenoviral Vectors

Recombinant, replication-deficient adenoviral vectors encoding E.coli β-galactosidase or having no insert (Adv0) were provided by Drs A. Byrnes and M. Wood (Oxford, UK). An adenovirus encoding porcine IκBα with a CMV promoter and a nuclear localization sequence (AdvIκBα) was provided by Dr R. de Martin (Vienna, Austria). Viruses were propagated in the 293) human embryonic kidney cell line and purified by ultracentrifugation through two cesium chloride gradients. The titers of viral stocks were determined through a plaque assay on 293) cells, as described (24).

### Infection Techniques

The elutriated human monocytes were incubated at approximately 2×106/ml in RPMI 1640 with 25 mM HEPES and 2 mM L-glutamine, supplemented with 5% (v/v) heat inactivated fetal calf serum and 10 units/ml penicillin/streptomycin. To optimize infection purified human monocytes were pretreated with M-CSF (100 ng/ml; obtained from the Genetics Institute, Boston Mass.) for 48 h to allow upregulation of integrin αVβ5, which has previously been shown to be essential for adenovirus infection of monocytes (25). The cells were then replated on 100 mm Petri dishes and infected for 2 h with a m.o.i. of between 10:1 and 120:1 (in most experiments, 20:1. 40:1 or 80:1 was used) of either AdvIκBα or Adv0, in serum-free RPMI 1640. Cells were then incubated in RPMI 1640 supplemented as above for 48 h to allow for significant over-expression of IκBα, as assessed. During the changes of medium involved, non-adherent cells were discarded, resulting in a further purification of monocyte-derived macrophages.

### Cytokine Analysis

For Northern analysis experiments, cells were replated at 5-0×106 cells per 100 mm Petri dish and stimulated with LPS (10 ng/ml), PMA (10 nM), zymosan (30 μg/ml), ionomycin (1 μM), or by UV irradiation (2000 J). After 4 h. cells were harvested, mRNA extracted and subjected to Northern analysis as in Buchan et al. (Clin. Exp. Imunol. 73 : 449 (1988).

In the assays for cytokine production, cells were replated at 5×105 cells per well on a 96-well dish, and stimulated as above for 4 or 16 h. Supernatants were analysed for TNFα (27), IL-1β, IL-6 and IL-8 (23), IL-10 (29, 30), IL-1ra and the p55 and p75 soluble TNF receptors (31) by ELISA. The proteosome inhibitor Cbz-Ile-Glu(O-t-Bu)-Ala-leucinal (PSI) was obtained from Calbiochem (Nottingham. England).

### Statistical methods

All statistical testing was performed using a paired comparison, one-sided Student's t test.

### RESULTS

Cytokine production in response to various stimuli.

LPS is capable of inducing all the cytokines (TNFα, IL-1β, IL-6, IL-8) and inhibitors (IL-10, IL-1ra and the soluble TNF receptors) assayed in this study (Table I; the results are means of 4-11 experiments). The induction of TNFα mRNA by zymosan, has previously been reported to be far weaker than LPS-induced TNFα mRNA in mouse peritoneal macrophages (32), but the TNFα response to zymosan in M-CSF-treated human monocytes was equal to that of LPS (Table 1).

LPS was a much stronger inducer of IL-1β than any other stimulus used. In agreement with observations in mouse macrophages Bondeson J. and Sundler R. (1995, Biochem, Pharmacol 50: 1753). IL-6 was induced equally well by LPS and zymosan. IL-8 could be induced by the entire array of stimuli. Zymosan and LPS were the only stimuli to induce IL-10 and the p55 soluble TNF receptor (Table I) whereas PMA could induce IL-1ra and p75 soluble TNF receptor.

### Does IkBa Over-Expression Inhibit LPS-Induced Pro-Inflammatory Cytokines?

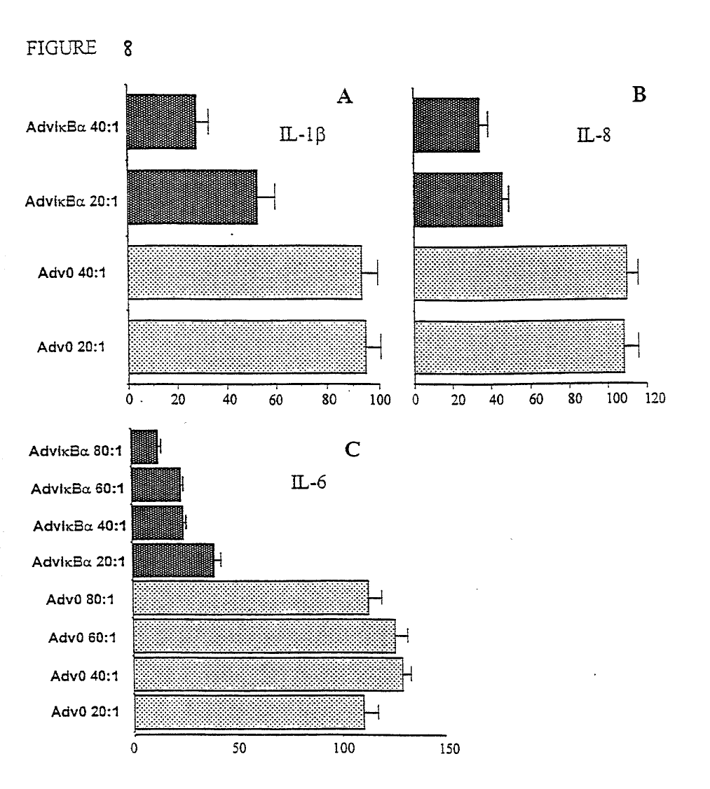

We previously observed that infection at a multiplicity of 20-80:1 with the IκBα adenovirus produced high levels of IκBα expression. This resulted in a potent inhibition of LPS-driven TNFα induction in human macrophages by blocking NFκB function (7). This was not due to loss of cells through apoptosis or other causes of cell death (7). The induction of L-10 and IL-8 by LPS also appears to be strongly NF-κB dependent (FIG. 8A-B), and there is potent inhibition of both these cytokines already at 20:1 of AdvIκBα.

TABLE I

| | Proinflammatory | | | | | | | |
|---|---|---|---|---|---|---|---|---|
| | cytokines (pg/ml) | | | | Anti-inflammatory cytokines (pg/ml) | | | |
| Stimulus | TNFα | IL-1 | IL-6 | IL-8 | IL-10 | IL-tra | P55 s TNF-R | P75 s TNF-R |
| LPS | 9000 | 4000 | 4000 | 4000 | 500 | 3000 | 100 | 1500 |
| Zymosan | 8000 | 200 | 4000 | 4000 | 300 | 2000 | 100 | 1000 |
| PMA | 6000 | 400 | 300 | 3000 | 0 | 1000 | 0 | 500 |
| UV light | 2000 | 200 | 100 | 3000 | 0 | 0 | 0 | 0 |
| Lomycin | 0 | 0 | 0 | 900 | 0 | 0 | 0 | 0 |
| TNFα | X | 200 | 2000 | X | 0 | X | X | X |

Results using supernatants harvested after 4 h of incubation were similar to those using 16 h of incubation (not shown). In contrast to the other cytokines studied here, there was a potentiation of IL-6 production of about 20-30 % in Adv0-infected cells, with LPS as well as with other stimuli (FIG. 8C), although adenovirus infection alone has no effect. Nevertheless, IL-6 expression was strongly inhibited by infection with AdvIκBα in human macrophages (FIG. 8C).

Since TNFα can induce the synthesis of other cytokines, e.g. IL-1β, IL-6 and IL-8, the potent inhibition by AdvIκBα might be secondary to inhibition of TNFα. Culturing with exogenous TNFα partly abrogated (just 20% inhibition with 100 ng/ml TNFα, compared with 62% inhibition with no cytokine) the effect of IκBα over-expression on IL-1β production (FIG. 9A), indicating at least a partial dependence on TNFα. However, similar experiments showed no major role for TNFα in LPS induced expression of IL-6 and IL-8 (results not shown).

To further investigate the signalling mechanisms involved. we sought to characterize whether TNFα-driven IL-1β might be less NF-κB dependent than the IL-1β response induced by LPS. One difficulty in doing this was the fact that TNFα is a weaker inducer of IL-1β in our system than LPS (Table 1). Yet the IκBα-induced inhibition of TNFα-induced IL-1β was relatively less potent than that seen with TNFα-induced IL-6 (FIG. 9B-C), as expected from the restoration of IL-1β synthesis by TNFα in cells infected with AdvIκBα.

### Does IkBa Over-Expression Affect LPS-Induced Anti-Inflammatory Cytokines?

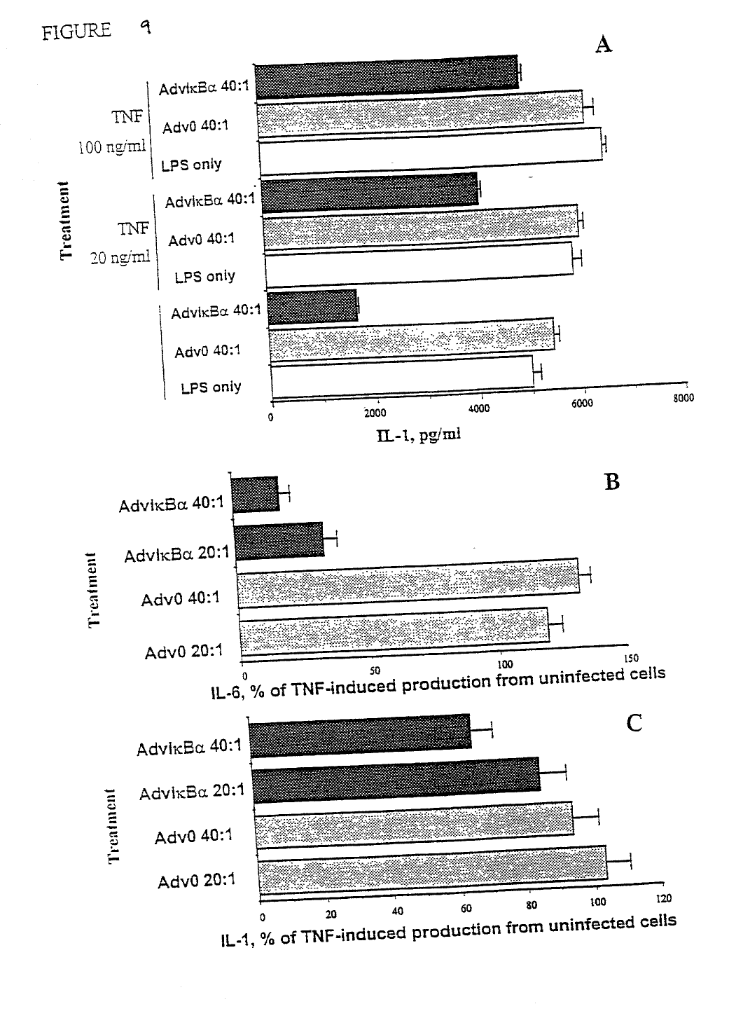

The major anti-inflammatory cytokine produced by macrophages is IL-10, in LPS-stimulated cells, there was gradual inhibition with increasing virus titers, but statistically significant inhibition (p<0.05) was only noted at 60:1 or 80:1 of AdvIκBα (FIG. 10A). This inhibition was still quite modest (30% at most), and since the human IL-10 promoter lacks κB sites, we investigated whether the inhibitory effects of AdvIκBα infection was indirect, via its effects on pro-inflammatory cytokines that are known to influence IL-10 expression. A combination of TNFα and LPS, considerably potentiated the IL-10 response and partially abrogated the inhibition by Adv1κBα (FIG. 10B). In contrast, IL-1 failed to have any significant effect (results not shown).

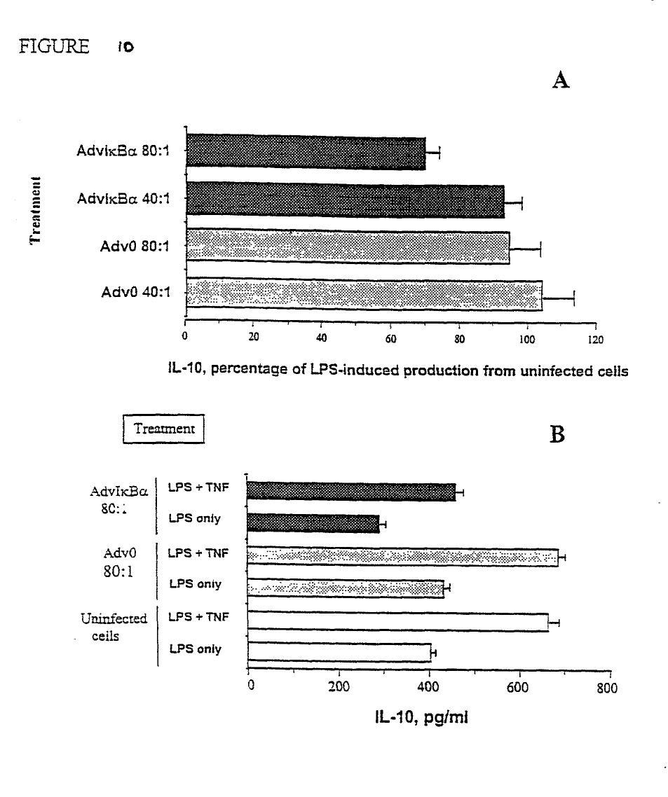

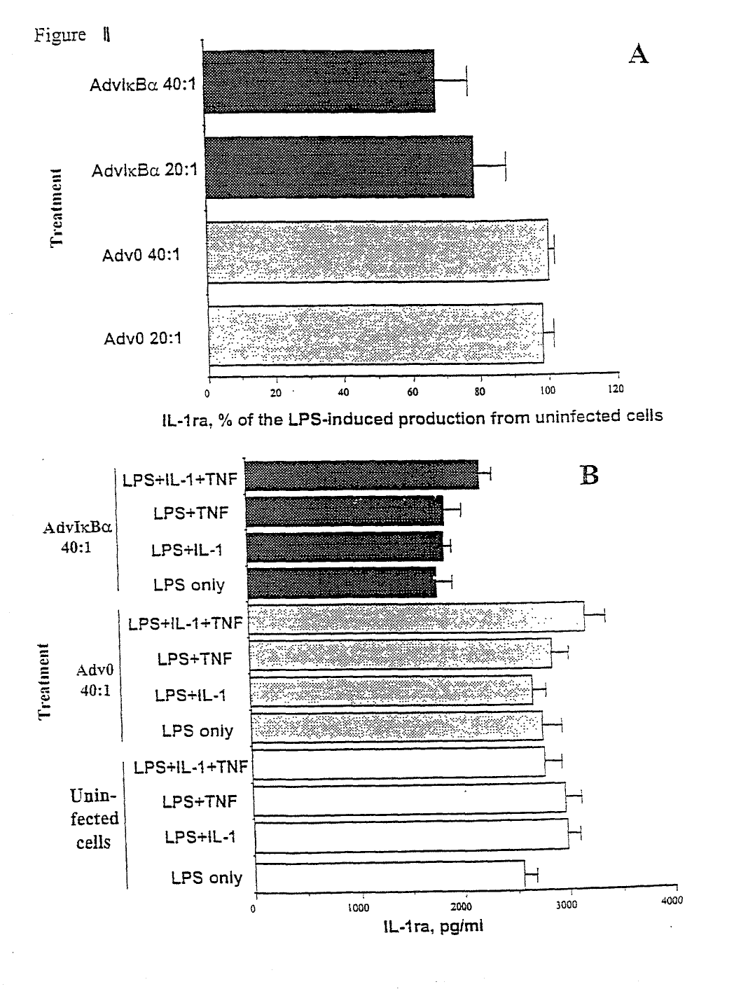

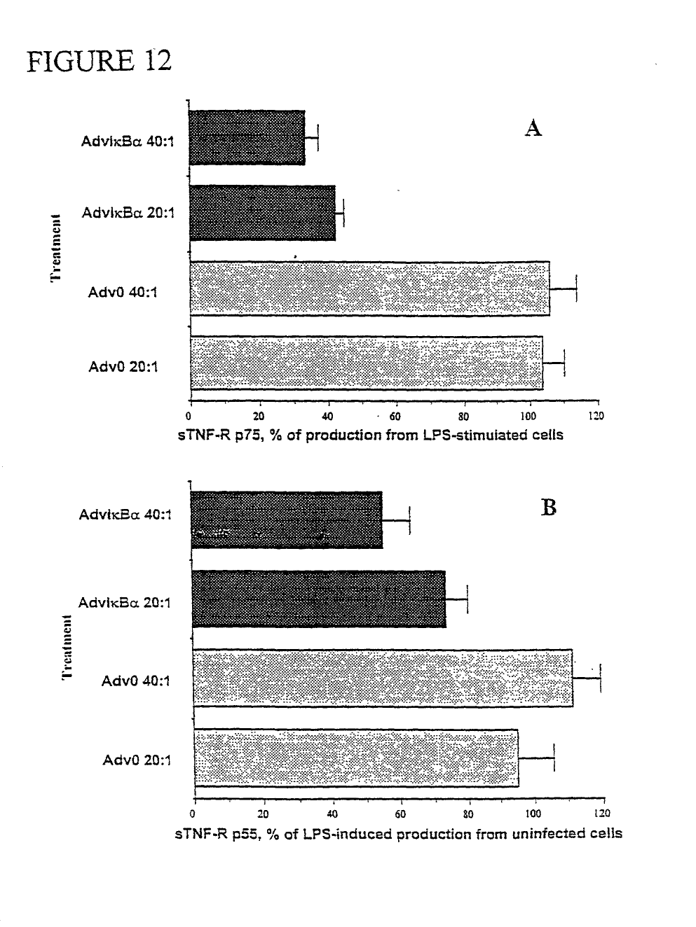

The LPS-induced production of IL-1ra was slightly inhibited (˜30%) by IκBα over-expression (FIG. 11A). This response was only modestly affected by adding back IL-1 and TNFα (FIG. 11B). In human macrophages LPS induces the production of the p75 soluble TNF receptor and to a lesser extent p55 soluble TNF receptor (Table I). Infection with AdvIκBα was observed to inhibit the LPS-induced production of both these soluble receptors (FIG. 12); this response was unaffected by adding back IL-1 or TNFα (results not shown).

### Does IkBa Over-Expression Inhibit Pro-Inflammatory Cytokines Induced by PMA or UV Light?

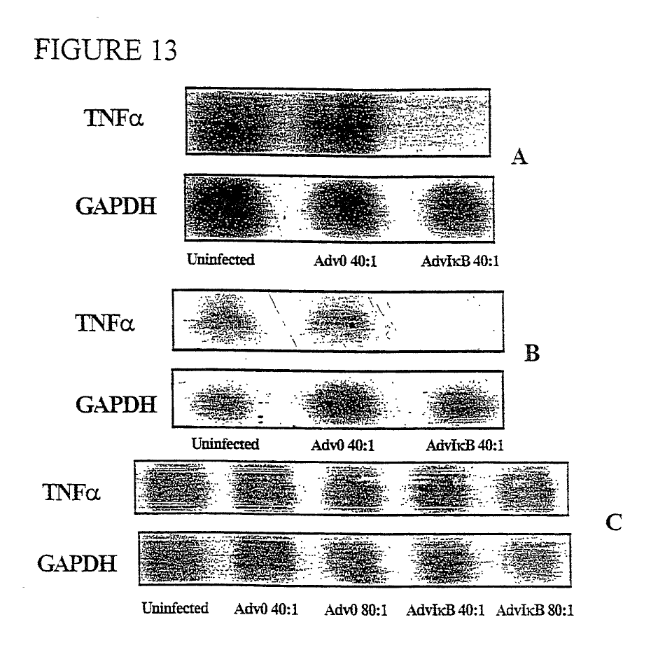

The induction of TNFα by PMA is very potently (˜90%) inhibited by IκBα over-expression, even more strongly than in LPS- (˜80%) or even UV light-stimulated cells (˜80%) (Table II). Again, results using supernatants harvested after 4 h of incubation were similar to those using 16 h of incubation (data not shown). A series of Northern analysis experiments demonstrated that, as for LPS-induced TNFα, the TNFα mRNA expression in response to PMA and UV light was ablated by IκBα over-expression (FIG. 13A-B).

As shown for TNFα, the induction of IL-1β and IL-6 by PMA or UV light was NF-κB dependent (Table II). Once again, the inhibition of IL-1β and IL-6 production by the AdvIκBα infection appears to be more potent in PMA-treated cells than in cells treated with LPS or UV light.

There was also inhibition (50-60 %) of the IL-8 response when cells infected with AdvlκBα were stimulated with PMA or UV light (Table II). Tonomycin, a stimulus that did not induce significant amounts of the other cytokines of interest induced a discernable IL-8 response, which was also inhibited by IκBα over-expression (data not shown).

TABLE II

| | | TNF-α | IL-1β | IL-6 | IL-8 | IL-tra | p75 s NF-R | p55 s TNF-R | IL-10 |
|---|---|---|---|---|---|---|---|---|---|
| PMA | Adv0 | 95.8 ± 3.3 | 120.9 ± 6.7 | 120.4 ± 10.9 | 124.5 ± 4.1 | 124.5 ± 7.8 | 11.02 ± 7.2 | ND | ND |
| | AdvlkBα | 10.4 ± 1.0 | 16.5 ± 0.8 | 8.5 ± 0.5 | 30.5 ± 2.0 | 60.8 ± 11.7 | 22.8 ± 2.4 | ND | ND |
| UV | Adv0 | 98.9 ± 2.3 | 102.2 ± 6.8 | 133.7 ± 8. | 108.5 ± 2.3 | 94.8 ± 8.8 | ND | ND | ND |
| light | AdvlkBα | 21.7 ± 0.9 | 40.2 ± 3.6 | 28.8 ± 2.6 | 54.5 ± 3.6 | 68.9 ± 5.9 | ND | ND | ND |
| Zym- | Adv0 | 97.5 ± 6.6 | 95.4 ± 9.8 | 143.6 ± 19.2 | 93.0 ± 4.0 | 108.0 ± 5.8 | 107.2 ± 8.4 | 123.6 ± 11.7 | 92.7 ± 7.2 |
| osan | AdvlkBα | 93.5 ± 6.4 | 92.8 ± 10.0 | 90.1 ± 12.9 | 94.5 ± 2.8 | 103.6 ± 9.1 | 90.3 ± 9.2 | 110.1 ± 6.4 | 101.4 ± 4.4 |

PMA did not induce detectable amounts of IL-10, but induced a detectable p75 soluble TNF receptor response, which was inhibited (75% at 40:1 of AdvIκBα) by IκBα over-expression, as was the PMA-induced production of IL-1ra (40% inhibition at 40:1 of AdvIκBα).

### Does IkBa Over-Expression Affect Zymosan-Induced Cytokines?

In contrast to the prior stimuli when the macrophages were activated with zymosan, infection with AdvIκBα had no effect whatsoever on TNFα protein (Table II) or mRNA expression (FIG. 13C), even at m.o.i of 80:1. The induction of IL-I and IL-8 by zymosan was also unaffected by the IκBα over-expression (Table II) but was a small (10-15% compared with uninfected cells) inhibition of IL-6. Zymosan-induced IL-10, IL-Ira (Table II) and the soluble TNF receptors (not shown) was also refractory to inhibition by IκB over-expression (Table I).

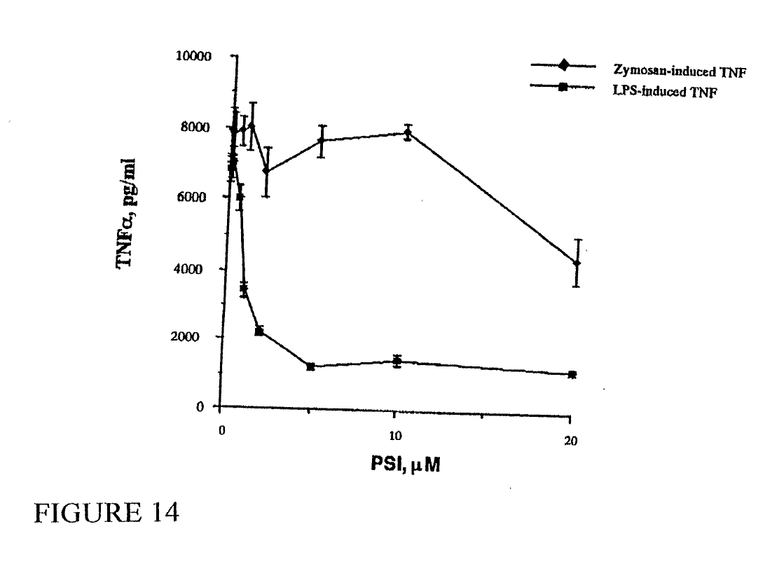

The independence from NFκB of zymosan induced TNF was further emphasised by studies with the proteosome inhibitor PSI. Inhibition of proteosome function inhibits IκBα degradation thus preventing NFκB nuclear translocation. PSI was very effective in blocking LPS induced TNF production but did not affect the response to zymosan (FIG. 14).

### DISCUSSION

The inventors have shown that adenoviral gene transfer into macrophages provides a reliable. reproducible and convenient method of studying intracellular signalling pathways. The natural inhibitor of NF-κB, IκBα was used to establish the principle. Transfer of the IκBα effectively inhibits NF-κB activity in human macrophages, mainly through the over-expression of IκBα inhibiting nuclear translocation of the p65/p5O subunits of NF-κB. This blocked LPS-induced TNFα both at the mRNA and the protein level. Even more interesting, it was found that the endogenous production of TNFα from rheumatoid synovial mononuclear cell cultures was also inhibited.

The fact that LPS-induced TNFα, IL-1β and IL-6 were all NF-κB dependent cytokines could be expected from the majority of data from murine cells and monocyte/macrophage cell lines. The results on LPS-induced cytokines also agree well with the results of Makarov et al (Gene Therapy (1997) 4 : 46) on LPS-induced IL-1β, IL-6 and IL-8 in monocytic THP-1 cells stably modified through retroviral gene transfer of IκB. Among the LPS-induced, pro-inflammatory cytokines studied here, IL-6 was most potently inhibited (>85%) by over-expression of IκBα whereas there was always some residual production of TNFα or IL-1β even in LPS-stimulated cells infected with high titers (120:1) of AdvIκBα (not shown). This may reflect a certain amount of preformed cytokine mRNA, but this could not be demonstrated in unstimulated cells (data not shown), and furthermore LPS, PMA or UV induced (FIG. 13) TNFα mRNA was profoundly downregulated by IκBα.

However it could not be excluded that this residual cytokine production emanated from the few uninfected cells still present, and work is in progress to elucidate this question using intracytoplasmic staining for cytokines. Another not exclusive hypothesis (discussed in more detail below), is that TNFα and other pro-inflammatory cytokines can be induced through both NF-κB dependent and NF-κB independent pathways.

Our finding that UV light induces a whole spectrum of pro-inflammatory cytokines in a NF-κB-dependent manner is novel. It is in contrast to the earlier report that UV-induced TNFα in RAW 264.7 cells does not involve NF-κB (Bazzoni, J. Clin. Invest. (1994), 93 : 56). Similarly, the finding that the PMA-induced induction of TNFα and other pro-inflammatory cytokines is profoundly downregulated by IκBα over-expression, disagrees with several earlier studies in stably transformed human cell lines (Makarov, Supra). In our hands, this stimulus was actually the one most strongly dependent on NF-κB, as judged by the percentage of inhibition, reproduced in seven separate experiments. These discrepancies between results obtained with human primary cells and those from various transformed cell lines indicate that, at least in some instances, the latter are questionable models for studying cytokine cell signalling occurring in primary cells, as is the case in vivo. In a way, this is not surprising, since there are interactions between the enzymes and transcription factors of the cell cycle machinery and the regulation of cytokine genes, e.g. Rb regulates ets, which is involved in cytokine activation (Bassuk A. and Leiden J. M. Adv, Immunol, (1997) 64: 65).

With regard to macrophage signal transduction, one of the most remarkable findings in the present study was that zymosan, although a very powerful macrophage activator, does not appear to require NF-κB for the induction of either pro- or anti-inflammatory cytokines. These findings would imply that there are, in human macrophages, both NF-κB dependent and NF-κB independent pathways of cytokine induction involved in the induction of TNFα and other pro-inflammatory cytokines. The modest (15%) of questionable significance inhibition of zymosan-induced IL-6 observed in cells over-expressing IκBα may reflect the observation that this cytokine was the most potently affected by IκBα over-expression. irrespective of stimulus (see Table II).

Another finding of importance is that in human macrophages, IL-10 is under complex control. and in LPS-stimulated cells, it appears to be at least partially driven via LPS-induced TNFα and IL-1. It is interesting to note that, even at 40:1 of AdvIκBα, when LPS-driven TNFα is abrogated by more than 60%, IL-10 is still not significantly inhibited (FIG. 10). At higher virus titers, resulting in even stronger inhibition of TNFα, there is some effect also on IL-10 but never, even with 80:1 of the virus, exceeding 30%. This is completely reversible by adding back TNFα, implicating that LPS-induced IL-10 is partly driven secondarily by TNFα. This finding agrees well with previous reports, and indicates that autocrine interactions can take place, even in short term (16 hour) cultures such as these.

Another intriguing finding was that the IκBα-induced inhibition of the LPS-induced production of IL-1β (FIG. 9A), but not the production of IL-6 or IL-8, or the soluble TNF receptors (data not shown), was also somewhat abrogated when TNFα was restored indicating that TNF induced IL-1β is mainly independent of NF-κB. This finding also may suggest that IL-1β is also, although to lesser extent than IL-10, driven partly by LPS-induced TNFα. This result echoes, although to a much more moderate extent, the previous work in RA joint cell co-cultures, where TNFα blockade was found to inhibit the production of IL-1, and subsequently of IL-6, IL-8, IL-10 and GM-CSF, which has led to the concept of a TNFα-dependent ‘cytokine cascade’ in inflammatory sites such as the rheumatoid synovium.

The dissection out of signalling pathways in normal primary cells is necessary as there are differences from cell lines (see above), and this is now possible within human macrophages, from either normal or pathological specimens, using this adenoviral technique. The model of human macrophages infected with AdvIκBα has, from a cytokine point of view, provided results similar to those from infecting human synovial cocultures, with the same virus (see below). The data suggest that NF-κB is an important therapeutic target in chronic inflammatory diseases, allowing profound downregulation of macrophage-produced pro-inflammatory cytokines, while not directly affecting the most important anti-inflammatory cytokines, IL-10 and IL-1ra. This would redress the disturbed equilibrium between these mediators.

### 3. DEMONSTRATION OF ADENOVIRUS INFECTION OF RHEUMATOID SYNOVIAL CELLS

Rheumatoid synovial cells produce high levels of cytokines and express high levels of integrins on their surface (Feldman M. et al. Ann. Rev. Immunol (1996) vol. 14). They have been used to demonstrate that in vivo activated cells, with spontaneous high levels of integrins and cytokines allow improved viral infection rates to be achieved. A use of such a system is also demonstrated.

### METHODS

Cells:

Synovium from patients with rheumatoid arthritis undergoing joint surgery was dissociated by cutting into small pieces, and digested with collagenase and DNAse I. The total cell mixture was cultured at 37° C. in RPMI 1640 with 25 mM HEPES and 2 mM L-glutamine, supplemented with 5% heat inactivated fetal bovine serum, on 24-well or 48-well plates.

### Adenoviral Vectors

Recombinant, replication-deficient adenoviral vectors encoding E. coli β-galactosidase (Advβgal) or having no insert (Adv0). An adenovirus encoding, porcine IκBα with a CMV promoter and a nuclear localization sequence (AdvIκBα) was provided as above. Viruses were propagated in the 293 human embryonic kidney cell line and purified by ultracentrifugation through two cesium chloride gradients. The titers of viral stocks were determined through a plaque assay on 293 cells. All viruses used were plaque purified from a master stock, in order to prevent contamination with wild form adenovirus.

### Infection Techniques

For experiments concerning cytokine or matrix metalloproteinase synthesis, freshly prepared rheumatoid synovial cells were resuspended in 1 ml serum-free RPM1 1640 on a 12-well plate at 2.5-4 million cells per well. After incubation for 1 h. they were either left uninfected, or infected with AdvIκBα or Adv0 at a multiplicity of infection of 40:1. After 2 h, the supernatants were removed and replaced with 0.5 ml RPM1 1640 supplemented with 5% heat inactivated fetal bovine serum. The nonadherent cells were carefully spun down and reintroduced to the rheumatoid cell coculture in 0.5 ml RPMI 1640 supplemented with 5% heat inactivated fetal bovine serum (total volume thus 1 ml.).

In some experiments, rheumatoid synovial cells were infected as above, but at a density of 0.4 million cells per well in a final volume of 0.2 ml, on a 96-well plate. Four wells of cells from each category—uninfected, Adv0-infected and AdvIκBα-infected—were plated. After incubation with adenovirus for 2 h, one well from each category was untreated, one treated with 20 μg/ml. of human recombinant IL-1 receptor antagonist (Cambridge Bioscience), one treated with 20 μg/ml. of the A2 anti-TNFα antibody (Centocor, Malvern, US), and one treated with both these cytokine inhibitors.

For infectibility experiments, cells were resuspended in 0.6 ml. serum-free RPMI 1640 on a 24-well plate at 1 million cells per well. After incubation for 1 h, they were either left uninfected, or infected with Advβgal or Adv0 at a multiplicity of infection of 40:1. After 2 h, the supernatants were removed and replaced with 0.4 ml RPMI 1640 supplemented with 5% heat inactivated fetal bovine serum. The nonadherent cells were carefully spun down and reintroduced to the rheumatoid cell coculture in 0.2 ml RPMI 1640 supplemented with 5% heat inactivated fetal bovine serum (total volume 0.6 ml).

### Analysis of Infectibility

Cells were scraped off the plates in the culture medium 48 h after infection, spun down and washed in FACS staining solution. Each batch of uninfected, Adv0-infected or Advβgal-infected cells were then resuspended in 25 μl staining solution and incubated with 125 ng of anti-CD3 PerCP and 500 ng of anti-CD 14 PE (both from Becton Dickinson, San Jose Calif.), in a total volume of 45 μl for 45 min at 4OC. They were then incubated at 37OC. for 10 min, before 45 μl of a 2 mM solution of Fluorescein di-(β-D-galactopyranoside (Sigma) was added for 1 min. Addition of excess (10×) ice-cold staining solution was used to stop the reaction. Cell fluorescence was analyzed by FACS.

### Analysis of Cytokines and Metalloproteases

In the assays for cytokine production, supernatants were taken off and nonadherent cells removed 48 h after infection. They were analysed for TNFα, IL-1β, IL-6 and IL-8, IL-10( ), IL-Ira and the p55 and p75 soluble TNF receptors by ELISA. The production of MMP-13 (Collagenase), MMP-3 (Stromelysin), MMP-13 and the tissue inhibitor of metalloproteases (TIMP-1) was analysed by ELISA kits purchased from Amersham (Little Chalfont, Bucks., UK).

In some experiments, aliquots of 50 μl were taken off after 12 h, 24 h, 48 h, 72 h and 110 h. After the nonadherent cells had been removed, and reintroduced to the cells in the same amount of medium, these aliquots were analysed for cytokines and metalloproteases as described above.

### RESULTS

### Infectibility of Rheumatoid Synovial Cells with Adenovirus

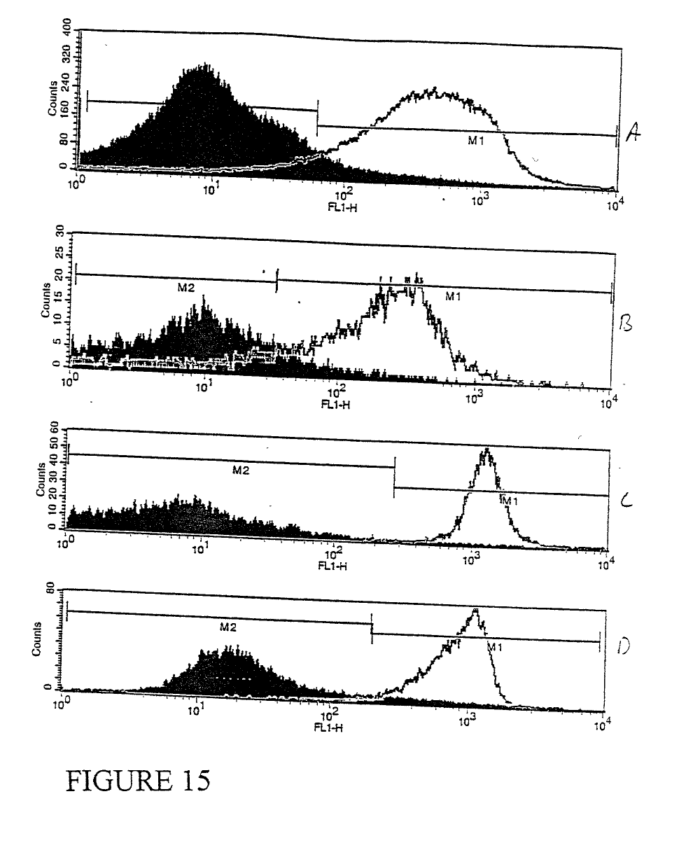

Infectibility of rheumatoid synovial cells was investigated using an adenovirus transferring the β-galactosidase gene (AdvβGal). Since 40:1 of AdvβGal infected 95-100% of human macrophages and 30:1 of AdvβGal infected nearly 100% of human fibroblasts, we decided to use 10:1I and 40:1 of AdvβGal in initial experiments. It turned out that 40:1 of AdvβGal infected nearly 100% of the entire rheumatoid synovial cell population (FIG. 15A), but that 10:1 infected only 60-70% (not shown), as evidenced by FACS analysis. All subsequent experiments thus use 40:1 of AdvβGal and other adenoviruses.

It was of interest to discriminate between the different cell types in the rheumatoid synovial cell cocultures, and to separately study the infectibility of the main constituents—macrophages, T lymphocytes and synoviocytes. This was performed through preincubating the cells with fluorescent antibody markers to CD3 and CD14, which allowed identification of CD3+/CD 14− (T lymphocytes), CD 14+/CD3− (macrophages) and CD3−/CD14− (synovial fibroblast) populations. By using human peripheral blood T cells and monocytes, and human skin fibroblasts, as controls, forward scatter/side scatter characteristics of these cell populations could be identified. Double gating was then used to ensure that the rheumatoid synovial cell populations chosen to represent T lymphocytes, macrophages and synoviocytes had the correct size/granularity and surface marker characteristics. As could be expected from earlier results, both macrophages (FIG. 15B) and fibroblasts (FIG. 15C) were easy to infect; more surprisingly in excess of 90% of the T lymphocytes were also infected by 40:1 of AdvβGal (FIG. 15D).

### IkBa Overexpression Inhibits the Production of Proinflammatory Cytokines

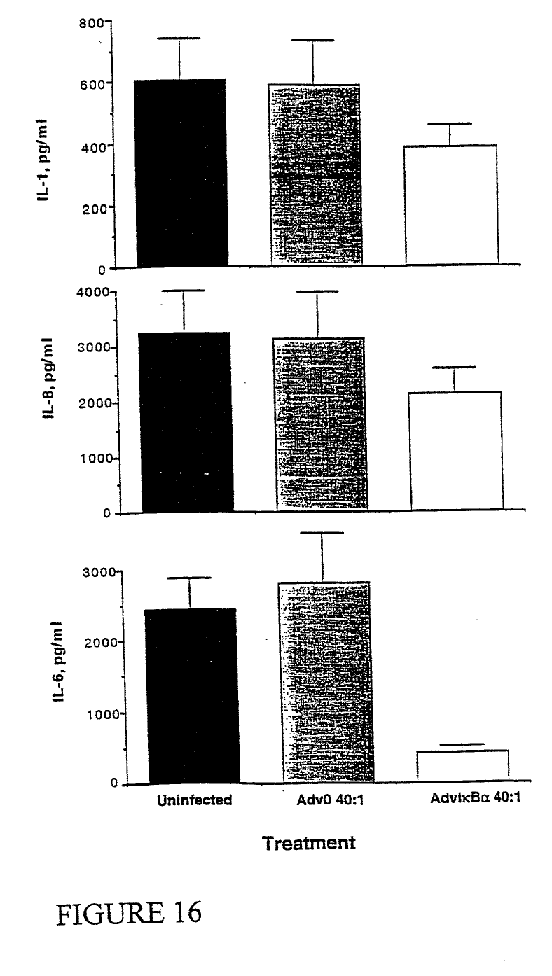

We have previously demonstrated that the spontaneous production of TNFα, as assessed two days after infection, was inhibited by 70% in cells infected with AdvIκBα, as compared with mock-infected cells or cells infected with Adv0. The spontaneous production of both IL-1β (FIG. 16A) and IL-8 (FIG. 16B) was also inhibited by IκBα overexpression, but to a more modest degree (40%, as compared with uninfected or Adv0-infected cells). In contrast, IL-6 was very potently inhibited (85%; FIG. 16C). These results agree with previous findings pointing out IL-6 as the most strongly NF-κB-dependent proinflammatory cytokine.

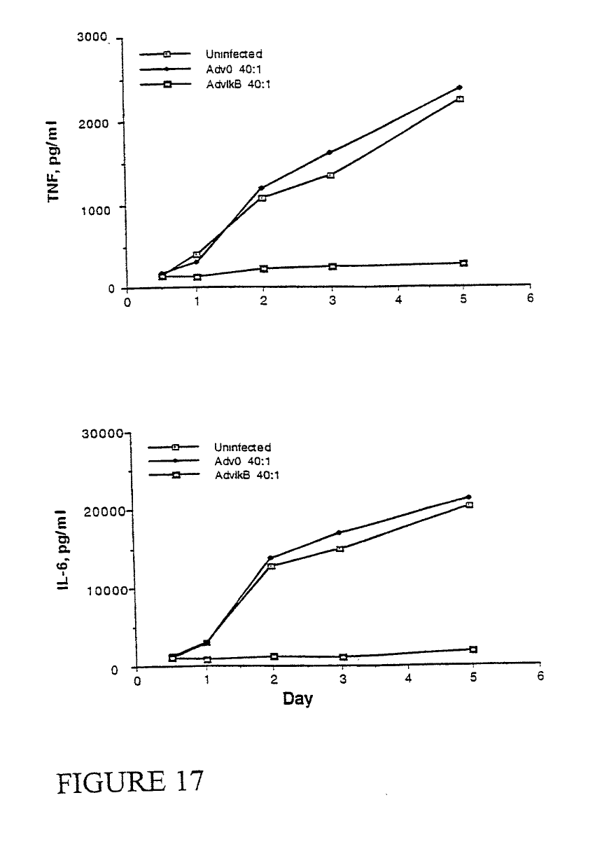

In a separate series of experiments, aliquots of medium were removed after 12 h, 24 h, 48 h, 72 h and 110 h, and cytokine analysis performed. It was seen (FIG. 17) that although the production of TNFα and IL-6 from uninfected or Adv0-infected cells gradually increased between day 1 and day 5, the cytokine production from AdvIκBα-infected cells remained almost static, indicating that once sufficient overexpression of IκBα had been achieved to inhibit NF-κB activation, only very little TNFα and IL-6 is produced.

### IκBα Overexpression does not Affect the Production of the Major Anti-Inflammatory Cytokines

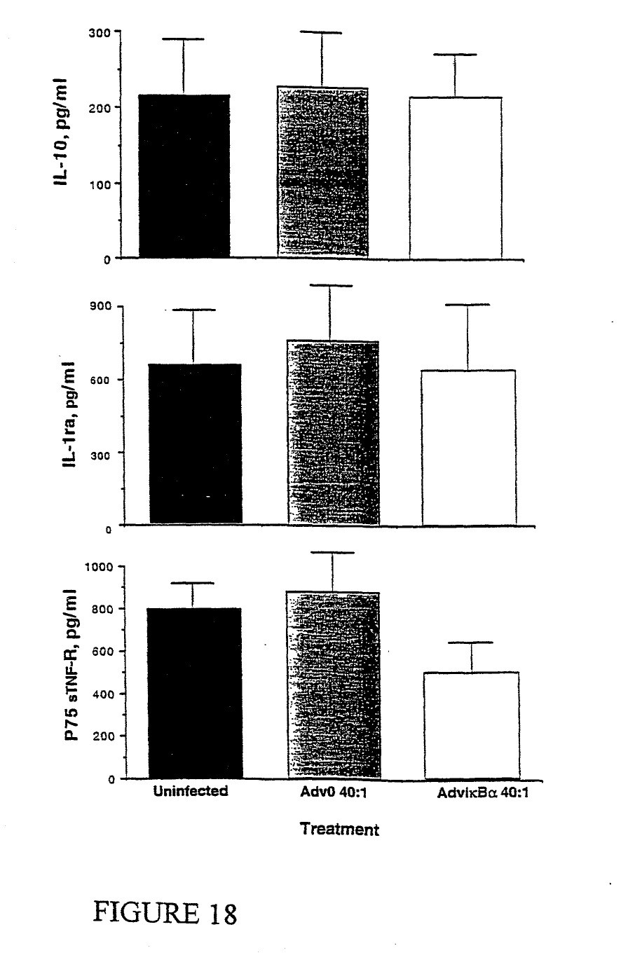

Previous results from human macrophages—major producers of IL-10 in the rheumatoid joint—have indicated that both IL-10 and the IL-1 receptor antagonist are NF-κB-independent in these cells, although the profound downregulation of TNFα and IL-1β induced by NF-κB downregulation causes some secondary diminuation. In rheumatoid synovial cell cocultures, the spontaneous production of both IL-10 and the IL-1 receptor antagonist are unaffected by overexpression of IκBα (FIG. 18A-B). The production of the p75 soluble TNF receptor is moderately (45%) inhibited, however (FIG. 18C).

### IκBα Overexpression Inhibits the Production of Metalloproteases

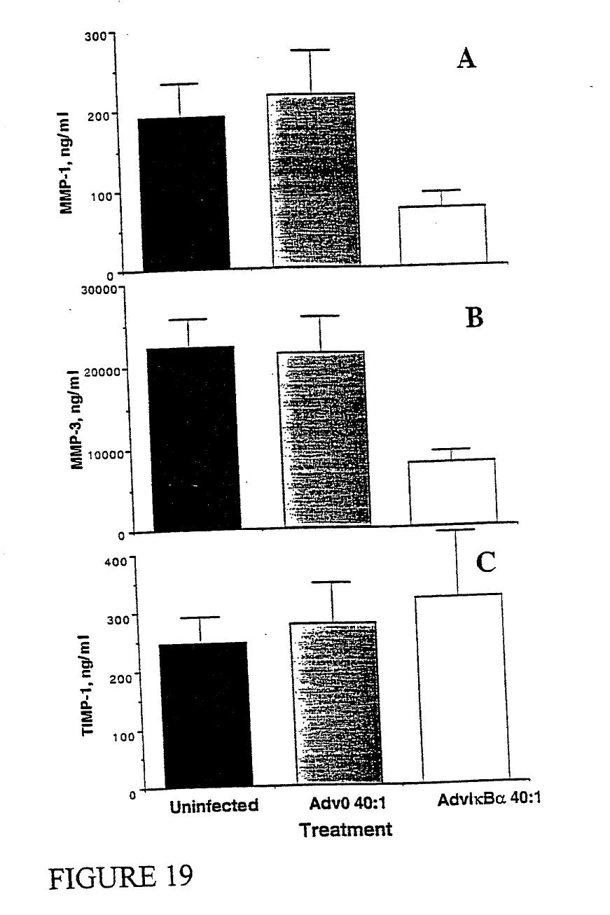

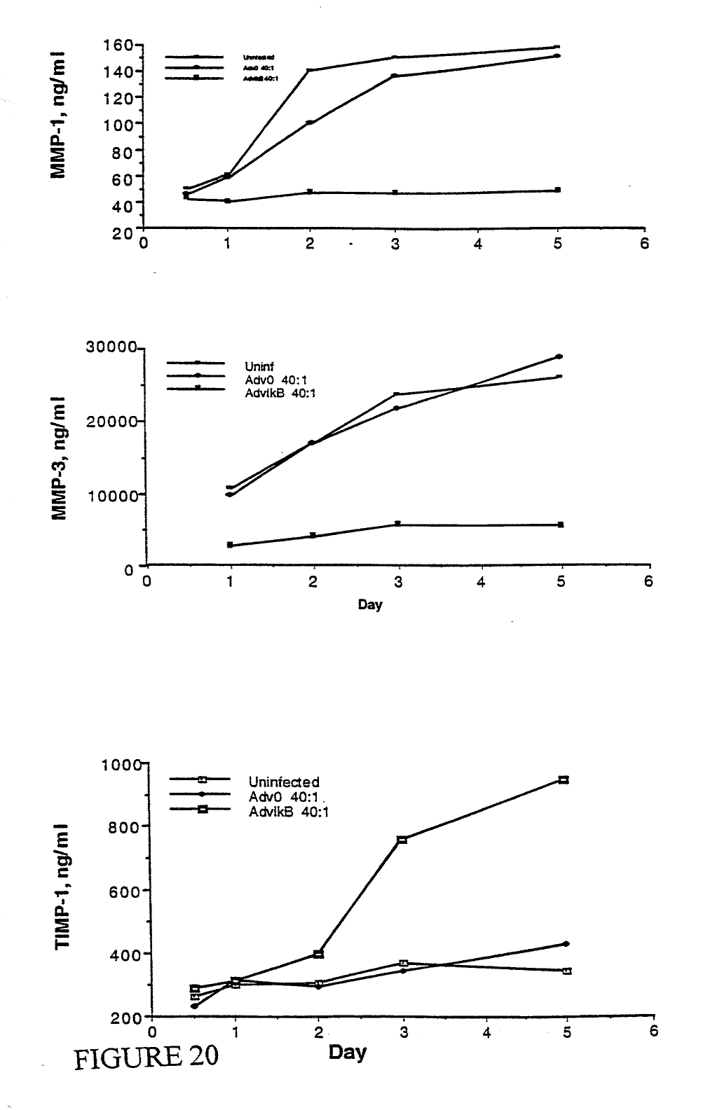

It is seen (FIG. 19A-B) that IκBα overexpression potently inhibits the spontaneous production of both MMP-1 and MMP-3 in rheumatoid synovial cells. In contrast, the spontaneous production of TIMP-1 is slightly potentiated after 2 days (FIG. 19C). Furthermore, while studying a timecourse of MMP-production (FIG. 20A-B), it appears that although uninfected and Adv0-infected rheumatoid synovial cell cocultures continue producing these enzymes throughout the incubation period, the AdvIκBα-infected cells produce very little of either MMP-1 or MMP-3 after significant IκBα overexpression has been achieved. Again, there is a remarkable contrast to the relatively strong potentiation of TIMP-1 production after 3 or 5 days (FIG. 20C).

These results indicate a direct dependence of NF-κB for the production of both MMP-1 and MMP-3, but not for TIMP-1. Both IL-1 and TNFα are known inducers of MMPs, however, and to rule out any influence of the downregulation of these two cytokines on MMP production, a series of experiments was performed with addition of IL-1ra and/or a TNF neutralizing antibody just after infection of cells. It was seen (FIG. 21) that the relative inhibition of MMPs by IκBα overexpression was largely unaffected by neutralization of either IL-1 or TNFα. This speaks in favour of the regulation of MMPs being directly NF-κB regulated; the inhibition of TNFα production in our cultures adds to it, since the MMPs are partially TNFα-driven, but the major part of the reduction of MMPs can be explained by a direct effect on their induction.

### DISCUSSION

These results strongly indicate the NF-κB is a key therapeutic target in rheumatoid arthritis, and probably other inflammatory diseases. The unique spectrum of effects achieved by inhibiting this transcription factor includes a potent inhibition of proinflammatory cytokines, particularly TNFα and IL-6, but no effect on the key anti-inflammatory mediators IL-10 and the IL-1 receptor antagonist. This would serve to readjust the disturbed balance between pro- and anti-inflammatory mediators in the diseased joint tissue. In addition, there is potent inhibition of the major destructive enzymes MMP-1 and MMP-3, but instead a potentiating effect on their major inhibitor, TIMP-1, indicating that NF-κB inhibition serves also to redress the disturbed balance between MMPs and their inhibitors.

1. An in vitro method for infecting one or more cells having an elevated integrin level with a viral vector capable of transporting exogenous or recombinant nucleic acid into the cells to be infected comprising the steps of:
a) collecting the cells to be infected; and
b) infecting the collected cells with a viral vector.

2. A method according to claim 1, wherein the cells are treated with at least one cytokine prior to infection with a viral vector.

3. A method according to claim 2 wherein the exogenous cytokine is one or both of GM-CSF and/or M-CSF.

4. A method according to any preceding claim wherein the cells are suspended and subjected to elutriation prior to collection.

5. A method according to claims 1 or 4, wherein the source of cells having an elevated cytokine level is selected from rheumatoid synovial cells, broncheoalveolar lavage from inflammatory respiratory diseases such as asthma and chronic obstructive respiratory disease, and gut biopsies of inflammatory bowel diseases such as Crohn's Disease and ulcerative colitis.

6. A method according to any preceding claim, wherein the cells are selected from one or more of macrophages, T-lymphocytes, synoviocytes, dendritic cells, chondrocytes, fibroblasts, epithelial cells and monocytes.

7. A method according to any preceding claim, wherein the viral vector used is an adenovirus.

8. A method according to claim 7, wherein the viral vector used is a replication-deficient adenovirus.

9. A method according to any previous claim wherein the m.o.i. is 10-1000.

10. A method according to any previous claim wherein the exogenous or recombinant nucleic acid is DNA.

11. A method according to any previous claim wherein the exogenous or recombinant nucleic acid comprises a nucleic acid sequence encoding a gene product of interest, operably linked to one or more regulatory sequences to enable the gene to be expressed within the infected cell.

12. A cell infected with a viral vector capable of transporting exogenous or recombinant nucleic acid into the cells infected obtainable by a method according to any preceding claim.

13. Use of an in vitro method or a cell according to any previous claim to study the effect of one or more exogenous compounds on the virally infected cell.

14. Use of a method according to any preceding claim for gene therapy.

15. A method of gene therapy comprising infecting one or more cells having an elevated integrin level with a viral vector containing exogenous or recombinant nucleic acid operably linked to a promotor into the cell to be treated.

16. Use of a viral vector containing exogenous or recombinant nucleic acid in the manufacture of a medicament to treat rheumatoid arthritis, inflammatory respiratory diseases such as asthma and chronic obstructive pulmonary disease, and inflammatory bowel diseases such as Crohn's disease and ulcerative colitis.
